(19) United States
(12) Patent Application Publication
(10) Pub. No.: US 2008/0221529 A1
(43) Pub. Date: Sep. 11, 2008

(54) Syringe with rear plunger lock

(76) Inventor: Bruce L. Kiehne, Queensland (AU)

Correspondence Address: Hoffman, Wasson & Gitler, P.C., Suite 522, 2461 South Clark Street, Arlington, VA, 22202, US

(21) Appl. No.: 11/713,645

(22) Filed: Mar. 5, 2007

(51) Int. Cl. A61M 5/00; A61M 5/31

(52) U.S. Cl. 604/211; 604/219

(57) ABSTRACT

A syringe having a plunger lock, the syringe comprising a syringe body having an open rear end, a plunger lock provided adjacent the open rear end, the plunger lock comprising at least one catch member having a portion extending partially into the open rear end of the syringe body and adapted to catch a portion of the plunger when the plunger has been pushed to the front of the syringe barrel, the plunger having a front end formed with a seal, and a rear end, and a projection adjacent the rear end which can ride underneath the at least one catch member when the plunger is inserted into the syringe barrel but which catches against the catch member upon attempted retraction of the plunger from the syringe barrel.

### FIELD OF THE INVENTION

This invention is directed to a syringe and typically a safety syringe that has a retractable needle and where the syringe has a rear plunger lock which locks the plunger in the fully forward position thereby making it difficult to retract the plunger for possible reuse of the syringe.

### BACKGROUND OF THE INVENTION

A medical syringe typically comprises a syringe body having a front nose portion and an open rear end. A needle can be attached in some manner to the front nose portion. A plunger is provided which extends through the open rear end. The plunger typically has an elongate plunger body, a plunger seal on the front of the plunger body and a thumb pad or something similar on the rear of the plunger body.

It is also quite well-known to provide a syringe having a retractable needle. One reliable needle retraction mechanism is described in many of our earlier patent applications, and a typical mechanism is described in our U.S. Pat. No. 6,994,690. Typically, when the plunger is pushed in a fully forward position, it triggers the retraction mechanism which causes the needle to be retracted back into the syringe body and typically into the plunger body.

It is however theoretically possible to pull the plunger back out of the syringe after retraction of the needle and then try to prise the needle out of the plunger for reuse. If the syringe does not have a needle retraction mechanism, it is quite easy to reuse the syringe by pulling the plunger back out of the syringe body.

Therefore, there would be an advantage if it were possible to provide some form of plunger lock to make it more difficult to retract the plunger after use. It would be especially preferred if the plunger lock was of a particular design to allow the plunger to be inserted through the open rear end of the syringe body and moved towards the front of the syringe, but still allowing the plunger to be retracted until such time as the plunger has been pushed fully forwardly at which time the plunger lock can engage to prevent retraction of the plunger. This allows a medical practitioner to adjust the volume of medicine.

It would also be advantageous if the plunger lock and a plunger had a complimentary design which means that rotation of the plunger does not allow the plunger to “bypass” the plunger lock thereby enabling the plunger to be removed from the syringe for possible reuse.

It will be clearly understood that, if a prior art publication is referred to herein, this reference does not constitute an admission that the publication forms part of the common general knowledge in the art in Australia or in any other country.

### BRIEF SUMMARY OF THE INVENTION

It is an object of the invention to provide a syringe with a plunger lock that may overcome at least some of the above-mentioned disadvantages or provide a useful or commercial choice.

In one form the invention resides in a syringe having a plunger lock, the syringe comprising a syringe body having an open rear end, a plunger lock provided adjacent the open rear end, the plunger lock comprising at least one catch member having a portion extending partially into the open rear end of the syringe body and adapted to catch a portion of the plunger when the plunger has been pushed to the front of the syringe barrel, the plunger having a front end formed with a seal, and a rear end, and a projection adjacent the rear end which can ride underneath the at least one catch member when the plunger is inserted into the syringe barrel but which catches against the catch member upon attempted retraction of the plunger from the syringe barrel.

Preferably two or more catch members are provided. Each catch member may comprise a small projection. The projection typically has a base portion attached to or formed integrally with the barrel and a free end portion extending into the barrel space. The projection may have a length of between 1-5 mm, and a width of between 1-5 mm and may be somewhat rectangular in configuration.

The projection on the plunger can abut against the free end portion of the projection that is part of the catch member upon attempted retraction of the plunger after the plunger has been pushed to the front of the barrel, thereby preventing retraction of the plunger.

The projection that forms part of the catch member can be deflected temporarily as the projection on the plunger is pushed past the catch member and the catch member can then return to its original position which can resist subsequent retraction of the plunger.

It is envisaged that the plunger lock will operate when the plunger has been fully pushed towards the front of the syringe body and any liquid in the plunger has been expelled from the syringe. However, there may be circumstances where it is desirable for the plunger lock to operate when the plunger is not fully pushed towards the front of the syringe body.

A front portion of the plunger (and typically the portion immediately behind the front seal) may be free from any full size longitudinal fins which usually run along the outside of the plunger body to provide a smooth sliding action of the plunger body in the syringe. This “smooth” portion may function to prevent the plunger from being inadvertently retracted too far (for instance when drawing up liquid into the syringe). This will be described in greater detail below.

The size of the syringe may vary and it is envisaged that the syringe will have a volume of between 1-20 millilitres. It is also envisaged that the syringe will be made of any suitable material such as plastic. The plunger may also be made of any suitable material and may have any suitable length and shape. Some suitable syringe sizes may be described in our earlier patent applications.

It is preferred that the syringe is a safety syringe which has a retracting needle, and a suitable retractable needle design may be similar to that described in our earlier U.S. Pat. No. 6,994,690, which is incorporated herein by cross reference. However, other retractable needle mechanisms may also be suitable. The plunger lock design according to the present invention may be suitable for various types of safety syringes and “conventional” syringes which do not have a retracting needle.

Any reference to a citation does not mean that this citation fomms part of the common general knowledge in Australia or elsewhere.

### BRIEF DESCRIPTION OF THE DRAWINGS

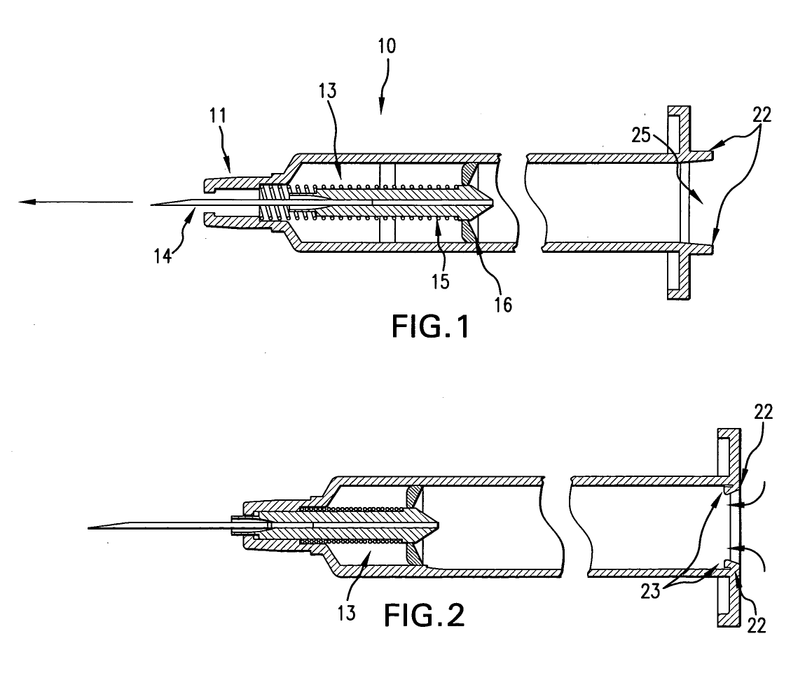

Two embodiments of the invention will be described with reference to the following drawings in which, FIGS. 1-4 illustrate the first embodiment and the remaining figures illustrate the second embodiment.

### DETAILED DESCRIPTION OF THE INVENTION

Referring initially to the first embodiment of the invention (FIGS. 1-4), there is illustrated a single use syringe having a retractable needle mechanism and a rear plunger lock.

The syringe has a syringe body 10 formed with a front nose portion 11 and an open rear end 12. A retractable needle mechanism (generally 13) can be fitted to the nose of the syringe body, and the needle mechanism is similar to that described in U.S. Pat. No. 6,994,690. The needle mechanism comprises a needle 14 which is fitted to a needle holder which comprises a larger inner part 15 and a small outer “ring” 16. A helical spring 17 extends about the larger inner part 15. The mechanism can be loaded through the open rear end 12 of the barrel and in FIG. 1, the mechanism (generally 13) has been inserted through the open rear end 12 of the barrel and is in the process of being pushed into the nose portion area 11.

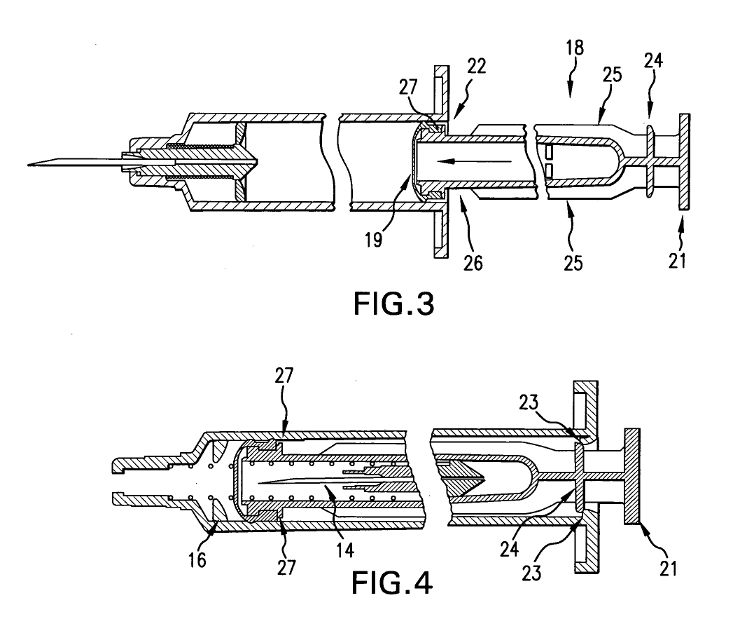

As illustrated in FIG. 2, the needle mechanism (generally 13) is in place and is held in the front part of the syringe. When a plunger 18 (see FIG. 3) is pushed into the open rear end of barrel 10 and towards the needle mechanism 13, when the front of the plunger pushes against the needle mechanism 13, it dislodges the outer “ring” 16 from the larger inner part 15, and the spring 17 extends to “shoot” the larger inner part 15 containing the attached needle 14 through the plunger seal 19 and into the confines of the plunger body. The retracted position is illustrated in FIG. 4. This mechanism is similar to that described in the above-mentioned US patent.

The rear open end 12 in this particular embodiment may contain a shroud [not illustrated] which extends about the open rear end 12 and, when the plunger has been pushed fully into the syringe barrel (see FIG. 4), the thumb pad 21 of the plunger sits within the confines of the shroud making it difficult for the thumb pad to be accessed for retraction of the plunger. However, the shroud does not comprise the rear plunger lock and is in addition to the rear plunger lock.

The rear plunger lock comprises cooperating members on the barrel 10 and the plunger 18. Referring initially to barrel 10, and particularly to FIG. 2, part of the rear plunger lock comprises a number of catch members 22. These catch members 22 initially form a small extension of the rear end of the syringe barrel (see FIG. 1), which are then pushed over (see the arrows in FIG. 2) to form the catch members. This enables the catch members to be formed in a simpler manner.

Each catch member has a base portion which is formed integrally with syringe barrel 10, and has a free end portion 23 which extends into the syringe barrel (see FIG. 2) and points towards the front of the syringe. Each catch member has a small amount of “spring” which means that it can be momentarily pushed inwardly and will then assume its natural position illustrated in FIG. 2. Each catch member is typically somewhat rectangular and has a length of between 1-5 mm and a similar width and may be seen as being “cantilevered” into the syringe barrel.

Plunger 18 contains a projection in the form of a, collar or ring 24 which is positioned in a rear part of the plunger and generally adjacent the thumb pad 21. Ring 24 has a diameter such that when the plunger is pushed into the syringe barrel (from the position illustrated in FIG. 3 to the position illustrated in FIG. 4), at some stage, ring 24 will pass underneath catch members 22 and momentarily deflect the catch members inwardly. When the plunger is in the fully forward position (FIG. 4) ring 24 has moved past the catch members 22 which can resume their natural position illustrated in FIG. 4.

Retraction of the plunger from the position illustrated in FIG. 4 is prevented as ring 24 now catches against the free ends 23 of each catch member 22.

Plunger 18 contains longitudinal fins 25 (which are quite conventional on plunges) and which function to facilitate a smooth action of the plunger. The fins 25 are designed and sized to glide against the inside wall of syringe body 10. Therefore, these fins will also deflect the catch members 22. To prevent these fins 25 from preventing proper functioning of the catch members, the fins 25 taper inwardly adjacent ring 24, thereby enabling proper locking of ring 24 against the catch members when the plunger is in the position illustrated in FIG. 4.

Similarly, the fins taper inwardly, or in the particular embodiment, are entirely absent from an area 26 which is immediately behind the plunger seal 19 (see FIG. 3). The rear of plunger seal 19 also contains a small extending ring 27 (best illustrated in FIG. 3) which also can pass and momentarily deflect the catch members and after ring 27 has passed the catch members, retraction of the plunger entirely out of the barrel is restricted. The advantage of this particular arrangement is that the plunger can be inserted into the barrel and pushed towards the front of the syringe (but not to the point where it triggers the retraction mechanism or to the point where ring 24 passes underneath catch members 23). The needle 14 can then be inserted into a vial (or something similar) and the plunger can be retracted to suck liquid from the vial into the syringe body. Inadvertent over retraction of the plunger (that is entirely out of the barrel) is prevented as ring 27 will engage with the catch members 22 to prevent this from occurring.

The plunger can then adopt the “injection” stroke where the plunger is pushed forwardly to eject liquid from the syringe and through the needle and this time, the plunger is pushed against the needle retraction mechanism which causes triggering of the needle retraction mechanism and retraction of the needle back into the plunger body. In this “fully forward” position, ring 24 has passed beneath catch members 22 which means that the plunger can no longer be retracted.

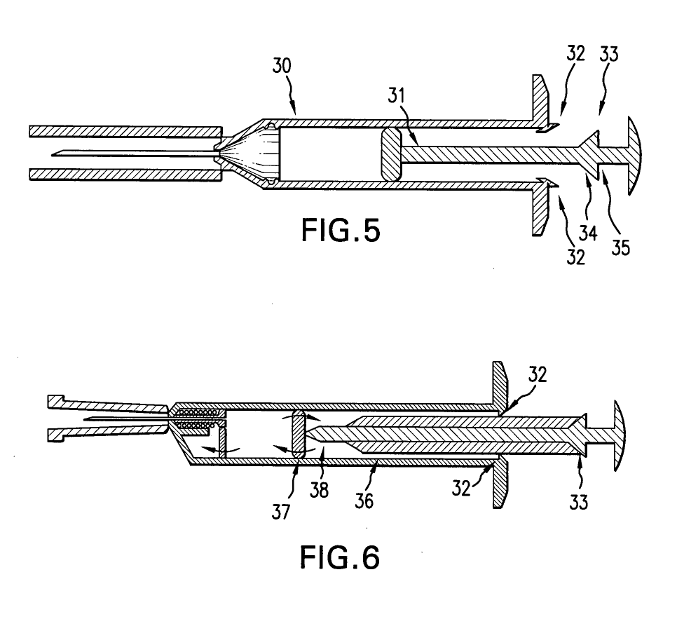

Referring to FIGS. 5-6, there is illustrated a second embodiment of the invention which is functionally similar to the first embodiment except that the design of some components is slightly different.

In the second embodiment, there is provided a syringe having a rear plunger lock which functions in a manner similar or identical to that of the first embodiment. Thus, the syringe comprises a syringe body 30 and a plunger 31. The open rear end of syringe body contains catch members 32 and a rear portion of plunger 31 contains a projection 33. Projection 33 has a forward ramped face 34 to facilitate passage of this part of the plunger underneath catch members 32 and behind ramped face 34 is an abrupt shoulder 35 which will strike catch members 32 when the plunger has been pushed towards the front of the syringe barrel and retraction of the plunger is attempted.

Referring to FIG. 6, the plunger contains longitudinal fins 36 which terminate before the plunger seal 37 to form a “smooth” portion 38.

Throughout the specification and the claims (if present), unless the context requires otherwise, the term “comprise”, or variations such as “comprises” or “comprising”, will be understood to apply the inclusion of the stated integer or group of integers but not the exclusion of any other integer or group of integers.

Throughout the specification and claims (if present), unless the context requires otherwise, the term “substantially” or “about” will be understood to not be limited to the value for the range qualified by the terms.

Any embodiment of the invention is meant to be illustrative only and is not meant to be limiting to the invention. Therefore, it should be appreciated that various other changes and modifications can be made to any embodiment described without departing from the spirit and scope of the invention.

1. A syringe having a plunger lock, the syringe comprising a syringe body having an open rear end, a plunger lock provided adjacent the open rear end, the plunger lock comprising at least one catch member having a portion extending partially into the open rear end of the syringe body and adapted to catch a portion of the plunger when the plunger has been pushed to the front of the syringe barrel, the plunger having a front end formed with a seal, and a rear end, and a projection adjacent the rear end which can ride underneath the at least one catch member when the plunger is inserted into the syringe barrel but which catches against the catch member upon attempted retraction of the plunger from the syringe barrel.

2. The syringe of claim 1, wherein two or more catch members are provided.

3. The syringe of claim 2, wherein each catch member comprises a small projection which has a portion attached to or formed integrally with the barrel and a free end portion extending into the barrel space.

4. The syringe of claim 3, wherein the projection has length of between 1-5 mm, and a width of between 1-5 mm.

5. The syringe of claim 1, wherein the projection on the plunger abuts against the free end portion of the projection that is part of the catch member upon attempted retraction of the plunger after the plunger has been pushed to the front of the barrel, thereby preventing retraction of the plunger.

6. The syringe of claim 5, wherein the projection that forms part of the catch member is deflected temporarily as the projection on the plunger is pushed past the catch member and the catch member then returns to its original position which resists subsequent retraction of the plunger.

7. The syringe of claim 1, wherein the projection on the plunger is a circular disk.

8. The syringe of claim 1, wherein plunger contains a projection immediately behind the plunger seal which catches with the catch members to resist retraction of the plunger entirely out of the syringe body.

9. The syringe of claim 1 which has a retractable needle.
